(19) United States
(12) Patent Application Publication
(10) Pub. No.: US 2009/0198127 A1
(43) Pub. Date: Aug. 6, 2009

(54) SYSTEM, DEVICE, AND METHODS FOR RESONANT THERMAL ACOUSTIC IMAGING

(75) Inventors: Jian Li, Gainesville, FL (US); Mark Sheplak, Gainesville, FL (US); Louis Cattafesta, Gainesville, FL (US); Henry Zmuda, Newberry, FL (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: UNIVERSITY OF FLORIDA RESEARCH FOUNDATION, INC., Gainesville, FL (US)

(21) Appl. No.: 12/089,962

(22) PCT Filed: Oct. 11, 2006
(86) PCT No.: PCT/US2006/039600

§ 371 Date: Sep. 4, 2008

Related U.S. Application Data
(60) Provisional application No. 60/726,036, filed on Oct. 11, 2005.

(51) Int. Cl. A61B 8/00; H02N 11/00

(52) U.S. Cl. 600/437; 310/300

(57) ABSTRACT

A thermal acoustic imaging (TAI) system includes a source of continuous amplitude-modulated RF or microwaves for irradiating a tissue region to be imaged, wherein a modulation frequency of the RF of microwaves resonantly excite the tissue region to emit thermal acoustic signals in response. The source preferably provides a substantially uniform power distribution in the region to be imaged. An acoustic transducer receives the thermal acoustic signals and generates an electrical signal in response. Matched filtering matched to a frequency of the amplitude-modulated RF or microwaves followed by delay-and sum or adaptive methods are preferably used to generate images from the electrical signals. The acoustic transducer is preferably a micro-electromechanical system (MEMS) transducer.

### FIELD OF THE INVENTION

The present invention is related to the field of thermal acoustic imaging, and, more particularly, to thermal acoustic imaging of biological entities for purposes such as medical diagnostics.

### BACKGROUND

Electromagnetically-induced thermal acoustic waves have been shown to be a viable mechanism for imaging certain cancers, especially human breast cancer. An electromagnetically-induced thermal acoustic image (TAI) can be produced by detecting ultrasound radiated by biological tissue that has been stimulated by the absorption of time-varying electromagnetic (EM) energy. When EM energy is absorbed by a tumor or tissue, the temperature of the tumor or tissue is raised. The increase in temperature causes a time-varying, thermally-induced mechanical expansion of, or vibration, in the tumor or tissue.

The time-varying, thermally-induced mechanical expansion, in turn, produces pressure waves that propagate throughout the tumor or tissue in different directions. Similarly, vibrating tissue characterized by a thermoelastically driven surface velocity radiates ultrasound waves that can be localized via acoustic measurements. The underlying physics of this thermal acoustic phenomenon in tissue is such that the thermally-induced acoustic signal is closely related both to the manner in which the increased tissue temperature is induced and to the absorption properties of the tissue within a particular tissue volume.

The spectral content of the incident EM radiation that induces thermoelastic tissue expansion and vibration ranges from low-energy radio frequencies (RF) to optical frequencies to higher energy ionizing radiation. Correspondingly, the wavelengths for conventional stimulating EM radiation for inducing thermal acoustic waves typically lie in the optical, infrared, microwave or RF range. At infrared and optical wavelengths, it can be difficult to stimulate a tumor at a depth of several centimeters or more owing to the effects of scattering and absorption. At RF and microwave wavelengths, however, radiation often can penetrate several centimeters into a tumor or tissue.

There are several distinct advantages to using electromagnetically-induced TAI for screening cancers such as breast cancer. These advantages stem directly from the inherent properties of TAI. Clinically, TAI does not ordinarily involve an invasive procedure, so there is less risk of infection and less discomfort for a patient. Research suggests, moreover, that TAI affords relatively higher sensitivity and specificity in comparison to other imaging or screening procedures. TAI also is generally viewed as being less expensive as well as a less risky procedure. In the specific context of screening for breast cancer, TAI is generally thought to provide particular advantages since it combines the benefits of EM stimulation and ultrasound imaging.

Nonetheless, the use of TAI for cancer screening has been hampered by several limitations. One such limitation relates to the transducers typically used with conventional TAI. The typical transducer is a piezoelectric ceramic-based ultrasonic transducer that is larger than the minimum acoustic wavelength generated for imaging. This typically results in a directional receiving response that, in turn, usually distorts the point spread function of the sensing array, thus limiting spatial resolution of the imaging.

Another limitation relates to the mode of EM excitation. Currently, EM excitation or stimulation proceeds with a sequence of pulses, the pulse repetition frequency usually being selected somewhat randomly without accounting for the elastic properties and acoustic radiation characteristics of the tumor that is being imaged. This obviates the opportunity for contrast enhancements via resonance excitation. More generally, the mode of conventional EM excitation typically prevents matched filtering, is generally ineffective for exciting tumor resonance, and for the most part, only provides limited bandwidth.

Still another limitation to using TAI for imaging tumors and screening for cancer also stems from the nature of EM stimulation utilized in conventional TAI-based imaging systems. With conventional systems, the EM field used to induce stimulation is a non-uniform EM field. A non-uniform EM field typically produces contrast gradients that yield uneven contrasts in an image and limits the depth that the field can penetrate into targeted tissue or tumors.

Moreover, since conventional systems generally utilize delay-and-sum reconstructions for generating images, the resolution and image quality provided is not satisfactory in certain applications, particularly when high levels of interference are present. These limitations need to be addressed if TAI is to become a viable alternative to more conventional technologies, such as analog and digital mammogram screening.

### SUMMARY OF THE INVENTION

The present invention is directed to a system, device and related methods for thermal acoustical imaging. Such imaging can be useful in screening for certain cancers and imaging tumors, particularly human breast tumors. In accordance with one embodiment of the invention, an image is acquired by inducing in a region of interest, such as one having or suspected of having a tumor, an acoustic resonant response, preferably using amplitude-modulated continuous RF or microwave radiation. Although not preferred, infrared and optical carrier wavelengths can also be used. The modulation frequency is selected to provide a resonant response in the region to be imaged. The response can include multiple acoustic wavelengths. Although described generally with regard to detection of breast tumors, the invention is in no way limited to breast cancer detection, or even cancer detection.

Like other relatively small compliant regions of tissue having a compressibility different than that of its surrounding medium, a tumor typically possesses several resonant frequencies. When the tumor is excited into resonance, an effective acoustic scattering cross section is increased significantly. The present invention can take advantage of this ability of biological tissues to resonate in response to a stimulating frequency, and can leverage this phenomenon to provide contrast-enhanced thermo-acoustic imaging of tumors.

A system according to one embodiment of the present invention is a thermal acoustic imaging (TAI) system for imaging a tumor. The system can include a resonant acoustic stimulator for inducing a resonant thermal acoustic stimulation in the tumor using an amplitude-modulated continuous RF or microwave radiation source. The radiation source preferably provides a substantially uniform power distribution in the region to be imaged. As defined herein, a “substantially uniform power distribution” is defined as a power distribution whose time-averaged intensity, which is proportional to the magnitude squared of the time-averaged intensity of the electric or magnetic field, does not deviate, in a least-squares sense, from some prescribed constant value over the spatial region that constitutes the volume being imaged. The prescribed constant value is generally less than 0.5 dB, and is preferably 0.1 dB, or less. The system also can include at least one micro-electromechanical system (MEMS) acoustic transducer for generating an electrical signal based on an acoustic response in the tumor when the tumor is stimulated by the resonant acoustic stimulator. An imager generates an image from the electrical signals, preferably using matched filtering that is matched to the AM source frequency and followed by conventional delay-and-sum or innovative adaptive methods to form images.

Another embodiment of the present invention is a method for imaging tumors. The method can include inducing a resonant thermal acoustic stimulation in the tumor using amplitude-modulated continuous RF or microwaves. Additionally, the method can include generating an electrical signal based on an acoustic response in the tumor, the acoustic response being generated by the amplitude-modulated continuous RF or microwaves.

Still another embodiment of the present invention is a machine-readable storage medium for use in imaging tumors with computer-based systems or devices. The storage medium can include computer instructions for inducing a resonant thermal acoustic stimulation in a tumor using an amplitude-modulated continuous electromagnetic wave. The storage medium further can include computer instructions for generating an electrical signal based on an acoustic response in the tumor when the tumor is stimulated by the amplitude-modulated continuous electromagnetic wave.

### BRIEF DESCRIPTION OF THE DRAWINGS

There are shown in the drawings, embodiments which are presently preferred. It is expressly noted, however, that the invention is not limited to the precise arrangements and instrumentalities shown in the drawings.

FIG. 1 is a schematic diagram of a system for imaging a tumor, according to one embodiment of the present invention.

FIG. 2 is a perspective view of two possible coil designs with current distributions optimized to produce a substantially uniform power distribution in the biological sample space.

FIG. 3 is a top planar view of the MEMS transducer embodied to include a plurality of acoustic transducers, according to another embodiment of the present invention.

FIG. 4 is cross-sectional view of one of the acoustic transducers illustrated in FIG. 3.

FIG. 5 is a flow chart of an exemplary method for imaging a tumor, according to still another embodiment of the present invention.

### DETAILED DESCRIPTION OF THE INVENTION

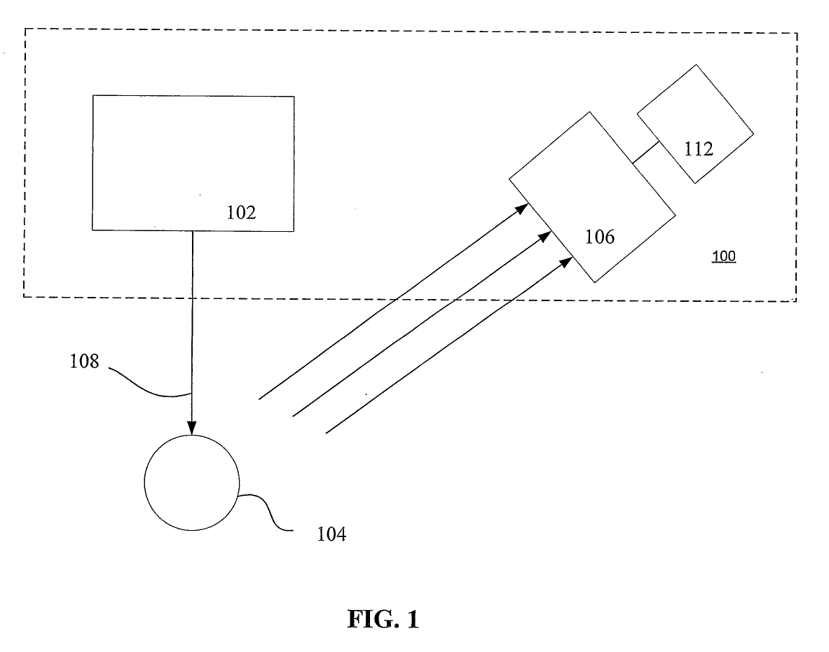

FIG. 1 is a schematic diagram of an exemplary system 100 for imaging a region of interest (e.g. a region of tissue suspected of having a tumor), according to one embodiment of the present invention. The system 100 is a thermal acoustic imaging (TAI) system. As used herein, the term “acoustic” pertains to mechanical vibrations transmitted by elastic medium, encompassing both audible sound waves and ultrasound waves, as well as the energy of sound waves.

The system 100 illustratively includes a source 102 of continuous, amplitude-modulated electromagnetic waves 108, preferably RF or microwaves, for inducing a resonant thermal acoustic stimulation in a tumor 104. The tumor 104 can be, for example, can be a tumor located in human breast tissue. The carrier frequency of the continuous, amplitude-modulated electromagnetic waves 108 is generally fixed, and is selected to achieve a desired tissue penetration and heat absorption response. The RF or microwave carrier frequency is nominally 100 MHz to 10 GHz. The modulation frequency is selected to excite a target structure in the region to be imaged, such as the tumor 104, and is preferably in a range that contains predicted resonant frequencies for a distribution of tumor sizes. The modulation frequencies are generally in the range from 100 kHz to 10 MHz, but can be higher or lower than this range.

As further illustrated, the system 100 also includes at least one transducer 106 for generating an electrical signal based on an acoustic response in the tumor when the tumor is stimulated by the source 102. The transducer is preferably a micro-electromechanical system (MEMS) acoustic transducer 106. Additionally, the system includes an imager 112, such as a CCD imager, which receives the electrical signal from the transducer 106 and forms an image therefrom.

The acoustic stimulation of the tumor 104 using an amplitude-modulated continuous RF or microwave 108 represents a significant divergence from approaches taken with conventional systems. One significant distinction lies in the fact that inducing resonant thermal acoustic stimulation in the tumor 104 allows the system 100 to take into account the mechanical properties of the tumor 104 as well as the effects that these properties have on a radiated acoustic field. Taking into account the mechanical properties of the tumor 104 affords advantages suggested by studies of the effects on the photo-acoustic response of isotropic spheres. Such studies have found that a radiated field is determined by the geometry and dimensions of a particle, as well as by its sound speed and density relative to a fluid that surrounds the particle.

Consistent with these results, in the context of cancer screening for breast cancer, for example, calculation of tumor resonant frequencies can be performed using common material properties of breast tissue and its response to ultrasound. Predicted resonant frequencies, ƒᵣₑₛ, can be expressed as a function of tumor radius a, and elastic modulus contrast ratio K=Eₜ/E, where Eₜ is the modulus of the tumor and E is the modulus of healthy tissue. It is expected that when a tumor is excited into resonance via EM simulation, the effective acoustic scattering cross-section could increase by a large factor, consistent with predictions for microsphere-based ultrasound contrast agents.

One of the advantageous results achieved with the system 100 is that the resonant frequency of the tumor 104 can be identified by maxima of the frequency response function between the modulation single-frequency and a system output, as described herein. The tumor response to the amplitude-modulated continuous electromagnetic wave 108, moreover, can facilitate matched filtering with the MEMS acoustic transducer 106 or other ultrasonic receiver so as to improve the signal-to-noise (SNR) and/or facilitate adaptive image formation for interference suppression.

The time delays of waves traveling between the tumor and the receiving transducers result in simple phase shifts among the transducer outputs. Robust adaptive image formation algorithms can be used to improve resolution, account for steering vector errors due to wave front distortions, and suppress interference. Optimal array design, with the goal of sparsely distributing acoustic transducers without sacrificing image quality, can also be used. The matched filter, matched to the known AM frequency, can be used to increase the transducer output SNR. The cancerous tumor responses are expected to vary, as a function of the AM frequency, differently from other benign or normal tissues. Robust adaptive algorithms can be used to exploit the unique data structure for image formation and for tumor detection.

In a preferred embodiment of the invention, the source of radiation 102 comprises a high uniformity electromagnetic field excitation system to provide a substantially uniform power distribution in the region to be imaged. The ideal fields for TAI systems are well specified. However, an advantageous arrangement of the multiple exciting sources to produce these fields has not been disclosed before. The most desirable field is one whose power density distribution is uniform over the entire imaging region extending to the chest wall. The challenges in realizing this distribution are twofold. First is the absorption of the RF energy resulting in attenuation, and secondly, the non-uniformity in the electrical properties of the breast. Conventional techniques typically utilize a single antenna that radiates the breast or an array of waveguide radiators. The former offers no ability to control the field at all, while the latter, though potentially able to influence the field pattern has not been thoroughly investigated.

Though different is several ways, EM coil design for Magnetic Resonance Imaging (MRI) can serve as a basis for improved systems that are able to provide uniform power distribution in the sample space. The use of a wire array structure is preferred for several reasons. Such structures can be adhered directly to the skin with an index matching gel, thus eliminating the mismatch at the air-skin interface. Wire structures are also relatively easy to fabricate, are conformable to the design of even exotic wire arrangements, and are lightweight and volume-efficient as compared to some other waveguide approaches - all of which make the wire structures more comfortable for the patient. Finally, the wire grid readily accommodates the integration of the MEMS acoustic sensors, and is generally inexpensive to produce.

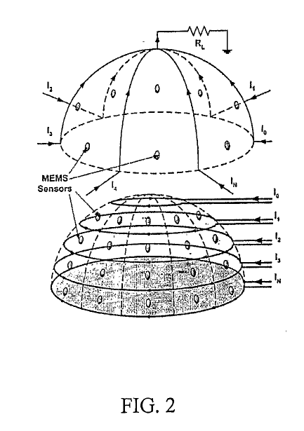

Two examples of possible configurations for the EM excitation system are shown in FIG. 2. The field is produced by radiating wires (or strips) currents, each a phasor whose amplitude and phase are chosen so as to optimize power uniformity. The RF/microwave operating frequency is chosen to minimize absorption in non-cancerous tissue while the modulation frequency is chosen to take advantage of thermal acoustic resonance and enhancing the measurement's signal-to-noise ratio. The power in the sample space is computed by the superposition of the EM fields produced by the wire array. In some cases the field can be obtained analytically, but in general the field can be accurately computed via a Method-of-Moments (MOM) approach.

The MEMS acoustic transducer 106 is preferably a piezoresistive transducer. Given that the function of the MEMS acoustic transducer 106 is to receive thermally-induced ultrasound in order to generate an electrical signal based on the acoustic response induced in the tumor 104 when the tumor is stimulated by the resonant acoustic stimulator 102, the MEMS acoustic transducer can be a non-reciprocal transducer.

In order to overcome limitations that are characteristic of conventional transducers in the context of TAI cancer screening, the MEMS acoustic transducer 106, as described herein, is a micro-machined transducer having sub-millimeter dimensions and enhanced bandwidth. More particularly, an aperture of size 2a, where a is the radius, is formed in MEMS acoustic transducer 106. a preferably lies in a range less than 0.5 mm. The MEMS acoustic transducer 106 preferably accommodates a backside-electrical contact scheme (not shown) that serves to isolate electrical contacts from the acoustic medium with which the system 100 is employed.

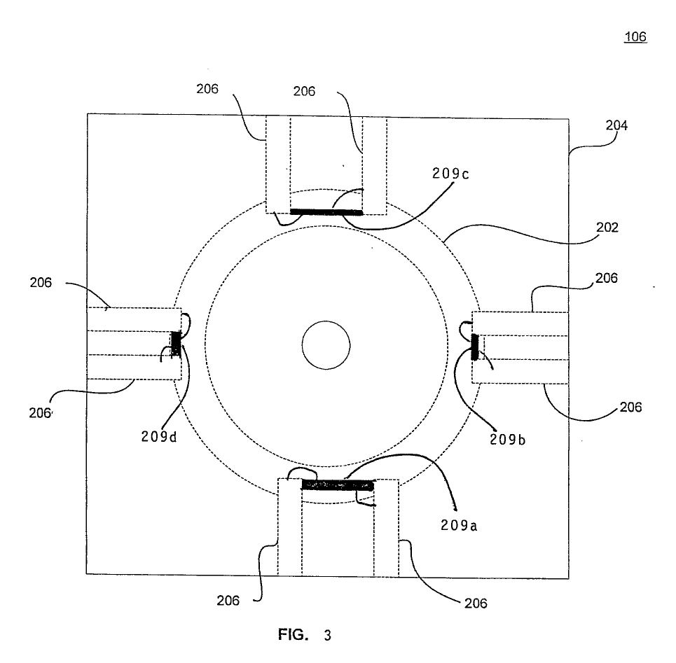

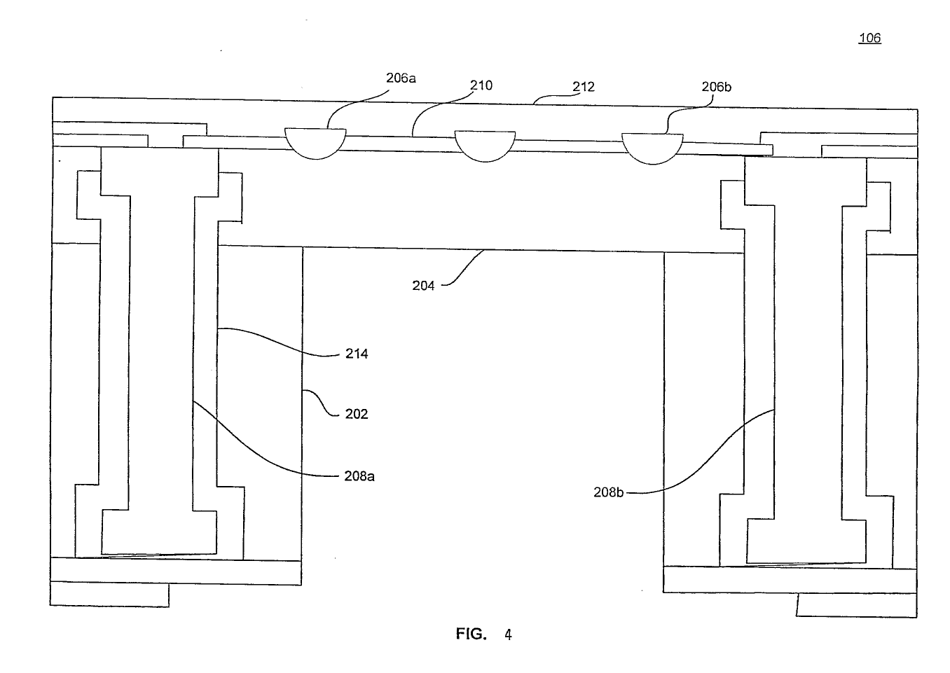

Referring additionally to FIGS. 3 and 4, the MEMS acoustic transducer 106, according to one embodiment, is schematically illustrated with a top planar view and cross-sectional view, respectively. As shown, the MEMS acoustic transducer 106 illustratively includes a substrate 204 and a composite diaphragm 202 disposed on the substrate. The MEMS acoustic transducer 106 further includes a plurality of piezoresistors 209a-d, each having a pair of leads 206 to transmit the electrical signal generated across the piezoelectric. Each piezoelectric 209a-d is positioned adjacent an edge of the composite diaphragm 202. In addition, as further illustrated in FIG. 4, a plurality of low-resistance, through-substrate electrical interconnects 208a, 208b extend through the annular substrate to the circular composite diaphragm 202.

In accordance with one embodiment, the composite diaphragm 204 of the MEMS acoustic transducer 106 is a circular composite diaphragm formed of silicon. Additionally, the MEMS acoustic transducer 106, as shown in FIG. 3, comprises a silicon dioxide layer 210 disposed on the composite diaphragm and a silicon nitride layer 212 disposed on the silicon dioxide layer. Silicon dioxide layer 214 is likewise disposed on each of the plurality of low-resistance through-substrate electrical interconnects 208a, 208b.

One or more of the plurality of piezoresistors of the MEMS acoustic transducer 106 can specifically be an arc resistor. Additionally, one or more of the plurality of piezoresistors of the MEMS acoustic transducer 106 can specifically be a tapered resistor. Indeed, according to one embodiment, the MEMS acoustic transducer 106 comprises four resistors—two arc resistors opposite one another and two tapered resistors opposite one another—each of the four resistors being equally spaced apart from the others and positioned at the edge of the composite diaphragm 202.

The MEMS acoustic transducer 106 can be fabricated, for example, in a complementary metal-oxide-semiconductor compatible process using deep reactive ion etching to produce high-aspect ratio through-wafer vias on a silicon-on-insulator wafer. Moreover, the plurality of low-resistance through-substrate electrical interconnects 208a, 208b can be formed of polysilicon and configured to facilitate a rugged “bump-bonded” sensor package with a flush top surface.

Although not required to practice the invention, a preferred MEMS fabrication sequence is provided below. Fabrication of the MEMS acoustic transducer 106 can begin with a double-sided polished n-type silicon-on-insulator wafer. Thermally grown silicon dioxide can be used to create a mask for the through-wafer via DRIE. The front and backsides of the wafer can be etched at approximately equal intervals. Etching through both sides of the wafer can be done to maintain the desired high aspect ratio of the structure. The oxide used for the mask can then be stripped using a buffered oxide etch. After the via formation, the interconnects can be dielectrically isolated from the bulk silicon substrate by growing a thermal oxide (e.g. 2 μm thick). Electrical conduction can be achieved through deposition of an LPCVD polysilicon layer over the oxide. This can be followed by boron diffusion doping of the polysilicon at a suitable annealing temperature and for a suitable time. After the low-resistance, through-substrate electrical interconnects 208a, 208b are planarized, the wafers can be patterned with the mask of the piezoresistors and implanted with boron to form P⁺ regions. A silicon dioxide layer can be grown to passivate the resistors. Subsequently, interconnect metallization and patterning can be performed. A low-stress nitride can then be deposited to form a moisture barrier and the diaphragm released via deep-reactive ion etching.

According to one embodiment of the invention, a signal processor processes the electrical signals generated by the MEMS acoustic transducer 404 using a robust adaptive image formation algorithm. The robust adaptive image formation algorithm can be implemented as a set of machine-readable instructions embedded in software code configured to run on the signal processor. Alternatively, the robust adaptive image formation algorithm can implemented with one or more hardwired dedicated circuits. In yet other embodiments the robust adaptive image formation algorithm can be implemented as a combination of machine-readable instructions and dedicated hardwire circuits. Moreover, the signal processor itself can alternatively be incorporated into or connected with the other elements of the system.

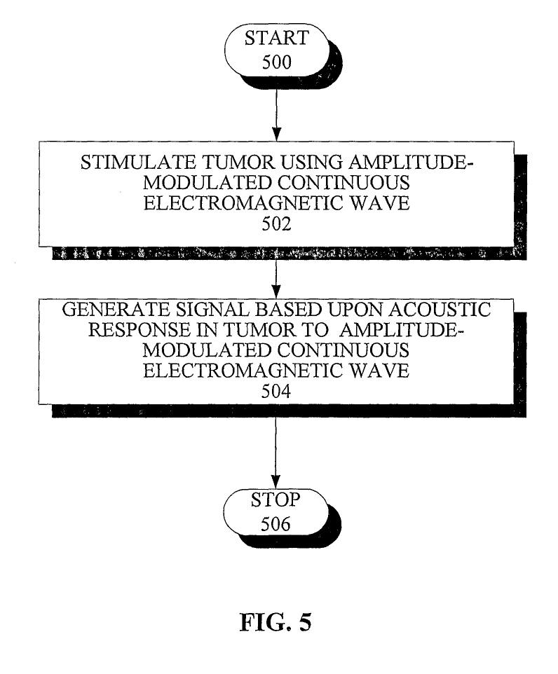

FIG. 5 provides a flow chart illustrating an exemplary method 500 for imaging tumors, according to yet another embodiment of the present invention. The method 500 illustratively includes, at step 502, inducing a resonant thermal acoustic stimulation in the tumor an using amplitude-modulated continuous RF or microwave, and, at step 504, generating an electrical signal based upon an acoustic response in the tumor to the amplitude-modulated continuous RF or microwave. The method illustratively concludes at step 506.

More particularly, according to one embodiment, a resonant thermal acoustic stimulation is induced by generating an electromagnetic (EM) field adjacent the tumor wherein the EM field has a uniform power density distribution over a region containing the tumor. According to still another embodiment, the amplitude-modulated continuous electromagnetic wave can have an operating frequency that reduces absorption in non-cancerous tissue adjacent the tumor. The amplitude-modulated continuous electromagnetic wave also can have a modulation frequency that increases a signal-to-noise ratio.

In a preferred embodiment, after matched filtering, conventional delay-and-sum or innovative adaptive methods are used to form images. Preferably, the image are formed using a robust adaptive image formation algorithm. For example, see U.S. Pat. No. 6,798,380 to Li (one of the present inventors), titled Robust Capon Beamforming, or related U.S. Pat. No. 6,894,642 also to Li, et al., titled Doubly constrained robust capon beamformer. The '380 patent discloses a method for enhanced Capon beamforming, referred to therein as an advanced robust Capon beamformer, which includes the steps of providing a sensor array including a plurality of sensor elements and wherein an array steering vector corresponding to a signal of interest (SOI) is unknown. The array steering vector is represented by an ellipsoidal uncertainty set. A covariance fitting relation for the array steering vector is bounded with the uncertainty ellipsoid. The matrix fitting relation is solved to provide an estimate of the array steering vector. Both the '380 patent and the '642 patent are hereby incorporated by reference into the present application in their entireties.

Illustratively, the method can further include determining for the amplitude-modulated continuous electromagnetic wave a modulation frequency range that contains at least one predicted resonant frequency in the region to be imaged. The resonant frequency can be predicted based on a distribution of tumor sizes. In addition, the method also can comprise identifying the at least one resonant frequency based upon a maximum response measured in the modulation frequency range tested.

According to still another embodiment, the method can further include determining at least one resonant frequency based on a radius of the tumor and an elastic modulus contrast ratio, the ratio being defined as K=Eₜ/E, where Eₜ denotes a modulus of the tumor and E denotes a modulus of healthy tissue.

The present invention can be realized in hardware, software, or a combination of hardware and software. The present invention also can be realized in a centralized fashion in one computer system, or in a distributed fashion where different elements are spread across several interconnected computer systems. Any kind of computer system or other apparatus adapted for carrying out the methods described herein is suited. A typical combination of hardware and software can be a general purpose computer system with a computer program that, when being loaded and executed, controls the computer system such that it carries out the methods described herein.

The present invention also can be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

This invention can be embodied in other forms without departing from the spirit or essential attributes thereof. The foregoing descriptions of preferred embodiments of the invention have been presented for the purposes of illustration and are not intended to exhaustive or to limit the invention. Modifications and variations are possible in light of the teachings disclosed herein, and it will be readily apparent to one skilled in the that the application is intended to cover adaptations and variations consistent with these teachings.

We claim:

1. A thermal acoustic imaging (TAI) system, comprising:
a radiation source of continuous amplitude modulated RF or microwaves for irradiating a tissue region to be imaged, wherein a modulation frequency of said RF or microwaves resonantly excites said tissue region to emit thermal acoustic signals in response thereto;
an acoustic transducer for receiving said thermal acoustic signals and generating electrical signals, and
an imager for generating an image from said electrical signals.

2. The system of claim 1, wherein said acoustic transducer comprises a micro-electromechanical system (MEMS) transducer.

3. The system of claim 2, wherein said MEMS transducer comprises a piezoresistive transducer.

4. The system of claim 2, wherein the at least one MEMS transducer has a diameter of less than one millimeter (1.0 mm).

5. The system of claim 1, wherein said MEMS transducer has a thickness of <five micrometers (5 μm).

6. The system of claim 1, wherein said radiation source comprises a plurality of radiating wires or strip currents, each a phasor whose amplitude and phase are selected to provide a substantially uniform power distribution in said region to be imaged.

7. A micro-electromechanical system (MEMS) acoustic transducer for use in imaging a tumor, the MEMS acoustic transducer comprising:
a substrate;
a composite diaphragm disposed on said substrate;
a plurality of piezoresistors adjacent an edge of the circular composite diaphragm; and
a plurality of low-electrical resistance through-substrate electrical interconnects extending through said substrate to the circular composite diaphragm.

8. The MEMS acoustic transducer of claim 7, wherein the composite diaphragm comprises silicon.

9. The MEMS acoustic transducer of claim 8, further comprising a layer of silicon dioxide layer disposed on the composite diaphragm and a silicon nitride layer disposed on said silicon dioxide layer.

10. The MEMS acoustic transducer of claim 8, wherein the plurality of piezoresistors comprises at least one arc resistor

11. A method of imaging, the method comprising the steps of:
inducing a resonant thermal acoustic stimulation in the region to be imaged using amplitude-modulated continuous RF or microwaves; and
generating an electrical signal based on an acoustic response in the region to be imaged responsive to said microwaves, and
forming an image from said electrical signal.

12. The method of claim 11, further comprising the step of matched filtering said electrical signal, and using delay-and-sum or innovative adaptive methods to form said image.

13. The method of claim 12, where said matched filtering is matched to a frequency of said amplitude-modulated RF or microwaves.

14. The method of claim 11, further comprising the step of determining a modulation frequency of said RF or microwaves based on a predicted resonant frequency in said region to be imaged.

15. The method of claim 14, wherein said modulation frequency is determined based on a distribution of tumor sizes.

16. The method of claim 14, further comprising the step of identifying said modulation frequency based upon measuring resonant responses from said region to be imaged for a range of modulation frequencies, and selecting a modulation frequency which provides a maximum resonant response.
